(19) United States
(12) Patent Application Publication
(10) Pub. No.: US 2024/0164418 A1
(43) Pub. Date: May 23, 2024

(54) ORGANIC SELENIUM ENRICHED EDIBLE MARINE MICROALGAL BIOMASS

(71) Applicant: Council of Scientific and Industrial Research (An Indian Registered Body Incorporated Under The, New Delhi (IN)

(72) Inventors: Arumugam Muthu, Thiruvananthapuram (IN); Reshma Ragini, Thiruvananthapuram (IN)

(21) Appl. No.: 17/773,356

(22) PCT Filed: May 19, 2021
(86) PCT No.: PCT/IN2021/050483

§ 371 Date: Apr. 29, 2022

(30) Foreign Application Priority Data
Jul. 8, 2020 (IN) 202011028987

(51) Int. Cl. A23L 17/00; A23K 10/16; A23K 20/158; A23K 20/20; A23K 20/22; A23L 33/12; A23L 33/16; C12N 1/12

(52) U.S. Cl. CPC A23L 17/65; A23K 10/16; A23K 20/158; A23K 20/22; A23K 20/30; A23L 33/12; A23L 33/16; C12N 1/12

(57) ABSTRACT

The present disclosure provides an organic selenium enriched edible marine microalga Nannochloropsis oceanica CASA CC201 with essential micronutrient Selenium, comprising organic selenium, along with omega 3 fatty acid and mono unsaturated fatty acid (MUFA). The present disclosure also provides a process for the organic selenium enrichment of edible marine microalga and a composition comprising the organic selenium enriched edible marine microalga Nannochloropsis oceanica. Moreover, the present disclosure also discloses a composition comprising the sustained bioprocess for organic selenium enriched edible marine microalga Nannochloropsis oceanica selenium enriched edible marine microalga, and wherein the composition is in the form of food, health, and feed supplements.

### FIELD OF THE INVENTION

The present disclosure relates to the field of nutraceuticals. Particularly, the present disclosure relates to an organic selenium enriched edible marine microalga Nannochloropsis oceanica CASA CC201 with essential micronutrient Selenium. More particularly, the present disclosure relates to a sustained bioprocess for obtaining the organic selenium enriched edible marine microalga for food and feed supplements.

### BACKGROUND OF THE INVENTION

Selenium is an essential trace element which is required for our body in very small quantity to perform various biological activities. Its deficiency may lead to many disorders like muscle disorders, thyroid disorders, brain disorders, cancer, inflammation etc. Dietary selenium supplementation is a solution for the above mentioned problem. Organic selenium sources are more digestible, absorbable and less toxic than inorganic sources. The marine microalga N. oceanica CASA CC201 has been fortified with organic selenium by sequential bioprocess with inorganic selenium over several generations. The organic selenium enriched marine microalgal biomass may be used as either food or feed additive for aqua or poultry feed industry.

Microalgae are one of the promising resources on earth since they are the reservoir of various bioactive compounds. They are rich with numerous high value molecules which include proteins, lipids, essential omega-3 fatty acids, carbohydrates, vitamins, pigments and other micronutrients. The commercial value of microalgae is exponentially rising day by day. They can be used for various purposes like food, health, feed additives, and cosmetics and also for energy production. They are capable of growing in any harsh conditions as well as less agriculture land and water consumed for their biomass production. Today, global population is fast growing and food scarcity, malnutrition, micronutrient deficiencies are becoming greater social issues; microalgae fortified with nutrients may be an appropriate solution for this problem. Microalgae enriched with micronutrients can be used as food or feed additives to address the above mentioned problem. The microalgae enriched with micronutrients are reported in various literature.

For instance, the Patent EP 2326708B1 discloses a novel method of selenium enrichment in photosynthetic microorganisms using organic selenium compounds like selenohydroxy acid. The said Patent also discloses that microorganism can efficiently enrich organic selenium from organic selenium compounds without the use of any inorganic selenium.

FR 2626890 discloses a method of preparation of algae, in which they were grown in fresh water medium under sterile conditions. One or more inorganic compounds and/or organic selenium were added to the culture medium. The said document also discloses the advantages of the method, such as: (i) it is cheap and easy; and (ii) no processing steps required; and (iii) since the method involved the step of growing algae in fresh water under sterile conditions, therefore contamination problems were excluded in the said method.

U.S. Pat. No. 6,197,295 discloses a production method as well as dried yeast products consisting of selenium. The organism used in the method was Saccharomyces boulardii sequela PY31. The method involved the step of adding sodium selenate to the medium for selenium enrichment. The yeast produced by the method had a final concentration of 4857 ppm selenium. The document also discloses the toxicity test of Se enriched yeast product by Rat Accute oral Toxicity model.

CZ 300809 discloses Scenedesmus quadricauda Sel V, which can be grown in presence of high concentration of selenate (Na₂SeO₄), which is toxic to the wild strain. S. quadricauda SelV showed growth comparable to the wild and also absorb significantly higher quantity of selenium, however, it showed toxicity to sodium selenite (Na₂SeO₃).

CZ 300808 discloses a Scenedesmus quadricauda strain which can grow in high concentration of both selenite and selenate.

CN 102925359 discloses a method of production of sea water Spirulina enriched with selenium. The method comprised the steps of adding selenium at a concentration of 1-160 mg/L in natural/artificial sea water.

CN 101715986 discloses a preparation method and application of selenium enriched Haemetococcus pluvialis which is rich in astaxanthin. The method of the said document comprised the steps of preparation of selenium enriched H. pluvialis by growing them in medium containing inorganic selenium continuously, wherein the selenium enriched H. pluvialis had a selenium content of 10 microgram/gram to 2000 microgram/gram with 99% of organic content.

As per the 2018 Global Nutrition report, current burden of malnutrition is unbearably high. Malnutrition is one of the socio economic problems in almost every country of the world. The world Health Organization (WHO) accounts that more than two billion people are suffering from micronutrient deficiency, globally. The Indian scenario is also not different. According to India's latest National Health Survey (NFHS-4, 2015-2016), more than half of Indian children are anaemic. Also, iodine deficiency, vitamin A deficiencies are also high. Essential trace element deficiency is often overlooked. Unlike macronutrient undernourishment, the health impacts of micronutrient deficiency are always not visible clearly.

One of the essential trace element nutrients is Selenium. Its deficiency is associated with various health issues. The main dietary sources of selenium are wheat, cereals, meat etc. Plants obtain selenium from soil where they are growing. pH and adverse climate changes like heavy flood largely affect the soil selenium status which in turn affect selenium content in plant based foods. Selenium intake was very low in some regions of China and Eastern Europe and it was found that the soil selenium status was very poor. The soil selenium status adversely affected the health of the people especially in China. Keshan disease is such an example. In India, some of the regions in Punjab and Himachal Pradesh have also reported for low soil selenium content. Also, the regions near Yamuna river where frequent flooding occurs, selenium content in soil reported to be low. Along with soil selenium status, refining and cooking will also decrease the selenium content in the food.

Even though there is a progress in addressing micronutrient deficiency, the pace is too slow. According to United Nations, if the current trend is following, SDG2 (second Sustainable Development Goal) of 2030 agenda will also be missed. To meet the target of SDG2 by 2030, the suggested strategies are: increased meat and dairy intake, reduction of supply chain losses and increased crop yields and products.

As the first scenario is considered, the productivity of poultry as well as fish is very crucial. Demands of both the markets are increasing largely. In India, the demand for eggs and chicken has been growing for the last two decades. India is one of the largest producers of broilers and eggs in the world. The Indian poultry market costed about INR 1494 billion in 2017. By 2023, the market is expected to reach INR 3,775 billion at a CAGR of 16.5% during 2018-2023. In the global poultry market also, India is considered as a fast growing poultry producer occupying a fourth position in terms of volume. The egg and broiler production in India during the year 2017 was around 75 billion and 4.2 million respectively. The production and consumption of eggs and broilers are showing an exponential growth in the present and will be the same in coming years. The change in food habits, urbanization, increasing awareness about balanced nutrition etc has driven the growth of poultry industry.

The global poultry feed market is worth of USD 3,161.14 million in 2017. It is expected to have a CAGR of over 3.91% over the forecast period. The forecast for Indian poultry feed market is CAGR of around 15% during 2015-2010.

Similarly, in India, the fish has worth INR 1,110 billion in 2018. It is expected to attain INR 1,998 billion in 2024, at a CAGR of 10.2% during 2019-2024. India contributes to around 6% of global fish products. During the past few years, the domestic consumption as well as fish and fish product export also increased tremendously. Due to food habit changes, and also the awareness of PUFA content and cholesterol reducing capacity of fish, led to raise the fish demand.

Since the fish production and consumption are increased, the aqua feed market is also exponentially increasing. The global aqua feed market is expected to progress form US$57.7 billion in 2012 to US$122.6 billion by 2019, at a CAGR of 11.40%. The Indian aqua feed market is worth of USD 1.20 billion in 2017 and is expected to have a CAGR of 10.4% during 2018-2023.

The emergence of various viral diseases like H1N1 which affects birds, pressurized the people to give more emphasis on animal welfare. Antioxidants like vitamin A, E and C, selenium, zinc etc, immunomodulators, acidifiers, and probiotics are suggested to be included in animal diet to boost their immunity. As a result of increased awareness about nutrition rich compound feed, regulatory policies by the government, more preferences are now there on feed and health aspects.

In these contexts, micronutrients enriched feed additives will serve a promising role in reducing nutrient deficiencies as well as improving the health of fish and poultry.

The poultry feed ingredients are generally classified into cereal grains, protein meals, fats and oils, minerals, feed additives and miscellaneous raw materials such as roots and tubers.

Cereal grains include wheat, corn, sorghum, barley, rye, triticale oats etc. Protein meals include both vegetable and animal sources such as oil seed meals, legumes, fish processing by-products. Fats and oils, generally called lipids are included in the diet to meet energy needs of the animals. They are also the sources of vitamins. Vitamins and minerals are essential for the body system of poultry which enhances their growth and development. Minerals like calcium and phosphorous are required in large quantity for bone formation and also for egg production. Other minerals like iron, copper, manganese, zinc, selenium, iodine and cobalt are required in milligrams but their deficiency will have led to severe health problems. Even though, the common diet contains vitamins and minerals, their demands are generally met through feed additives.

On average, marine microalga Nannochloropsis oceanica biomass is composed of available carbohydrates (37.6% w/w), crude protein (28.8%) and total lipids (18.4%). Ca, K, Na, Mg, Zn, Fe, Mn, Cu, Ni, Co are the minerals present in it. Toxic heavy metals like Cd and Pb are present in negligible amount. Poly unsaturated fatty Acids (PUFA) like [symbol] -3 fatty acids (EPA) are also high in N. oceanica. Accordingly, there is a need in the art to obtain an enriched edible microalga with organic selenium of about 99% of organic selenium being converted upon inorganic selenium supplementation.

### OBJECTIVES OF THE INVENTION

The main objective of the present disclosure is to provide an organic selenium enriched edible marine microalga for food and feed supplements.

Another objective of the present disclosure is to provide a sustained bioprocess for obtaining the organic selenium enriched edible marine microalga for food and feed supplements.

Yet another objective of the present disclosure is to provide a process for obtaining the organic selenium enriched edible marine microalga, wherein the process bio-convert more organic selenium (more than 99%) by withstanding up to 1000 μM sodium selenite from beginning (Day1) without any lag in its growth.

Another objective of the present disclosure is to provide a process for bio accumulating more organic selenium, which is more bioavailable and readily digestible by animals.

Another objective of the present disclosure is to provide a process for obtaining the organic selenium enriched edible marine microalga, wherein the process comprises obtaining the organic selenium enriched edible marine microalga from sea water, without freshwater usage, thereby, making the process an economically viable one.

### SUMMARY OF THE INVENTION

In an aspect of the present disclosure, there is provided an organic selenium enriched edible marine microalga Nannochloropsis oceanica CASA CC201.

In another aspect of the present disclosure, there is provided a process for obtaining an organic selenium enriched edible marine microalga Nannochloropsis oceanica CASA CC201 as described herein, wherein said process comprises: treating N. oceanica CASA CC201 with an inorganic selenium having a concentration in the range of 100-500 μM over several generations for obtaining an organic selenium enriched edible marine microalga Nannochloropsis oceanica CASA CC201.

In another aspect of the present disclosure, there is provided a composition comprising an organic selenium enriched edible marine microalga Nannochloropsis oceanica, as described herein.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

The detailed description is described with reference to the accompanying figures. In the figures, the left-most digit(s) of a reference number identifies the figure in which the reference number first appears. The same numbers are used throughout the drawings to reference like features and components.

FIG. 1 depicts growth study of N. oceanica with respect to inorganic selenium treatment, in accordance with an embodiment of the present disclosure.

FIG. 2 depicts intracellular selenium content quantification in N. oceanica by ICP MS, in accordance with an embodiment of the present disclosure.

FIG. 3 depicts morphology analysis of Wild N. oceanica and Se acclimatized N. oceanica, in accordance with an embodiment of the present disclosure.

FIG. 4 depicts growth study of selenium acclimatized N. oceanica in fresh medium with and without inorganic selenium and comparison of biomass productivity, in accordance with an embodiment of the present disclosure.

FIG. 5 depicts intracellular selenium content quantification in selenium acclimatized N. oceanica grown in fresh medium with and without inorganic selenium by ICP MS, in accordance with an embodiment of the present disclosure.

FIG. 6 depicts growth of high selenium tolerant N. oceanica with respect of control in terms of biomass productivity, in accordance with an embodiment of the present disclosure.

### DETAILED DESCRIPTION OF THE INVENTION

The invention will now be described in detail in connection with certain preferred and optional embodiments, so that various aspects thereof may be more fully understood and appreciated.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

As used herein, the term “omega 3 fatty acid” refers to the essential polyunsaturated fatty acid, whose structure is characterized with presence of a double bond at the third atom from the terminal methyl group. In the present disclosure, the term “omega 3 fatty acid” includes but not limited to eicosapentaenoic acid (EPA).

The term “GCMS” herein refers to gas chromatography-mass spectrometry method performed to analyse the fatty acid content in the selenium enriched microalga. This analytical method is performed to evaluate the content of monounsaturated fatty acid (MUFA) and polyunsaturated fatty acid (PUFA), such as eicosapentaenoic acid, linoleic acid and arachidonic acid, in the selenium enriched Nannochloropsis oceanica CASA 201.

The term “SEM” herein refers to the morphological analysis performed using scanning electron microscope at 15 kV voltage and 1500 KX magnification. The cover slip with sample of a substance is placed on a metal stub and then sputter coated with gold. This is an analytical method performed to evaluate the surface morphology of a substance. In the present disclosure, the morphology of the wild and selenium acclimatized N. oceanica was analyzed by SEM.

The term “ICP MS” herein refers to the inductively coupled plasma mass spectroscopy method performed to determine elements at ultra-low concentration in biological fluids. In the present disclosure, ICP MS is performed to analyse the amount selenium accumulated in the microalga cultures sample.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a concentration in the range of 700 μM to 1000 μM should be interpreted to include not only the explicitly recited limits of about 700 μM to about 1000 μM but also to include sub-ranges, such as 750 μM to 910 μM, and so forth, as well as individual amounts, within the specified ranges, such as 730 μM, and 890 μM.

Marine microalga Nannochloropsis oceanica biomass is composed of available carbohydrates (37.6% w/w), crude protein (28.8%) and total lipids (18.4%). Ca, K, Na, Mg, Zn, Fe, Mn, Cu, Ni, Co are the minerals present in it. Poly unsaturated fatty Acids (PUFA) like [symbol] -3 fatty acids (EPA) which are also high in N. oceanica. Along with these, in the present disclosure, Nannochloropsis oceanica CASA201 is enriched with trace element Selenium. So, the enriched edible marine microalga Nannochloropsis oceanica can be used as a nutrient supplement in various feed formulation like poultry, aqua, cattle, pet feeds as well as multivitamin food supplements.

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided an organic selenium enriched edible marine microalga Nannochloropsis oceanica CASA CC201.

In an embodiment of the present disclosure, there is provided an organic selenium enriched edible marine microalga Nannochloropsis oceanica CASA, comprises at least 99% of organic selenium, along with omega 3 fatty acid in the range of 9-12% and monounsaturated fatty acid (MUFA) in the range of 33-35%.

In an embodiment of the present disclosure, there is provided an organic selenium enriched edible marine microalga Nannochloropsis oceanica CASA, comprising at least 99% of organic selenium, along with omega 3 fatty acid in the range of 9-11%, or 9-10%, and monounsaturated fatty acid (MUFA) in the range of 33-35%, or 33-34%.

In an embodiment of the present disclosure, there is provided an organic selenium enriched edible marine microalga Nannochloropsis oceanica CASA as described herein, wherein the organic selenium enriched edible marine microalga Nannochloropsis oceanica CASA further comprises EPA content of 9-12%. In another embodiment of the present disclosure, the organic selenium enriched edible marine microalga Nannochloropsis oceanica CASA further comprises EPA content of 9-11%, or 9-10%.

In an embodiment of the present disclosure, there is provided an organic selenium enriched edible marine microalga Nannochloropsis oceanica CASA CC201, comprises at least 99% of organic selenium, along with omega 3 fatty acid wherein the EPA content in the range of 9-12% and MUFA in the range of 33-35%.

In an embodiment of the present disclosure, there is provided a process for obtaining an organic selenium enriched edible marine microalga Nannochloropsis oceanica CASA CC201 as described herein, comprises treating N. oceanica CASA CC201 an inorganic selenium having a concentration in the range of 100-500 μM over several generations for obtaining an organic selenium enriched edible marine microalga Nannochloropsis oceanica CASA CC201.

In an embodiment of the present disclosure, there is provided a process for obtaining an organic selenium enriched edible marine microalga Nannochloropsis oceanica CASA CC201 comprising at least 99% of organic selenium, along with omega 3 fatty acid wherein the EPA content in the range of 9-12% and MUFA in the range of 33-35%, the process comprises treating N. oceanica CASA CC201 an inorganic selenium having a concentration in the range of 100-500 μM over several generations for obtaining an organic selenium enriched edible marine microalga Nannochloropsis oceanica CASA CC201.

In an embodiment, the present invention provides a process for obtaining the organic selenium enriched edible marine microalga Nannochloropsis oceanica CASA CC201 as described herein, said process comprising: (i) treating N. oceanica CASA CC201 an inorganic selenium having a concentration in the range of 100-500 μM over several generations for enrichment of organic selenium; (ii) transferring N. oceanica CASA CC201 of step (i) to a fresh medium with inorganic selenium and without inorganic selenium to know whether it can really tolerate the high inorganic selenium; and (iii) enriching N. oceanica CASA CC201 which can withstand the highest concentration of inorganic selenium in the same manner as enriched in step (ii) for obtaining an organic selenium enriched edible marine microalga Nannochloropsis oceanica CASA CC201.

In an embodiment, the present invention provides a process for obtaining the organic selenium enriched edible marine microalga Nannochloropsis oceanica CASA CC201 as described herein, wherein said process optionally comprises transferring N. oceanica CASA CC201, which can tolerate maximum inorganic selenium, to more high inorganic selenium concentration in the range of 700-1000 μM to enrich the intracellular organic selenium content and to enhance biomass productivity

In an embodiment of the present disclosure, there is provided a process for obtaining an organic selenium enriched edible marine microalga Nannochloropsis oceanica CASA CC201 as described herein, wherein inorganic selenium is preferably sodium selenite.

In an embodiment of the present disclosure, there is provided a process for obtaining an organic selenium enriched edible marine microalga Nannochloropsis oceanica CASA CC201 as described herein, wherein the said process comprises: (i) treating N. oceanica CASA CC201 with different concentrations in the range of 100-500 μM of inorganic selenium over several generations for enrichment of organic selenium; (ii) transferring N. oceanica CASA CC201 of step (i) to fresh medium with inorganic selenium and without inorganic selenium to know whether it can really tolerate the high inorganic selenium; (iii) repeatedly, enriching N. oceanica CASA CC201 which can withstand the highest concentration of inorganic selenium in the same manner as enriched in step (ii); and (iv) optionally, transferring N. oceanica CASA CC201, which can tolerate maximum inorganic selenium, to more high inorganic selenium having a concentration in the range of 700-1000 μM to enrich the intracellular organic selenium content and to enhance biomass productivity, wherein inorganic selenium is preferably sodium selenite.

In an embodiment of the present disclosure, there is provided a process for obtaining an organic selenium enriched edible marine microalga Nannochloropsis oceanica CASA CC201 as described herein, wherein the organic selenium enriched edible marine microalga Nannochloropsis oceanica CASA is cultured in natural sea water.

In an embodiment of the present disclosure, there is provided a process for the organic selenium enrichment of edible marine microalga Nannochloropsis oceanica CASA CC201 as described herein, said process comprising: (i) treating N. oceanica CASA CC201 with different concentrations in the range of 100-500 μM of inorganic selenium, wherein inorganic selenium is preferably sodium selenite, over several generations for enrichment of organic selenium; (ii) transferring N. oceanica CASA CC201 of step (i) to fresh medium with inorganic selenium and without inorganic selenium to know whether it can really tolerate the high inorganic selenium; (iii) repeatedly, enriching N. oceanica CASA CC201 which can withstand the highest concentration of inorganic selenium in the same manner as enriched in step (ii); and (iv) optionally, transferring N. oceanica CASA CC201, which can tolerate maximum inorganic selenium, to more high inorganic selenium concentration in the range of 700-1000 μM to enrich the intracellular organic selenium content and to enhance biomass productivity, wherein said inorganic selenium is sodium selenite, and wherein the organic selenium enriched edible marine microalga Nannochloropsis oceanica CASA is cultured in natural sea water.

In an embodiment of the present disclosure, there is provided a composition comprising an organic selenium enriched edible marine microalga Nannochloropsis oceanica as described herein.

In an embodiment of the present disclosure, there is provided a composition of organic selenium enriched edible marine microalga Nannochloropsis oceanica, wherein the organic selenium enriched edible marine microalga Nannochloropsis oceanica CASA CC201, comprises at least 99% of organic selenium, along with omega 3 fatty acid in the range of 9-12%, and Monounsaturated fatty acids (MUFA) in the range of 33-35%.

In an embodiment of the present disclosure, there is provided a composition of organic selenium enriched edible marine microalga Nannochloropsis oceanica as described herein, wherein the organic selenium enriched edible marine microalga Nannochloropsis oceanica is present in the range 10-30% (w/w).

In an embodiment of the present disclosure, there is provided a composition of organic selenium enriched edible marine microalga Nannochloropsis oceanica as described herein, said composition is used in a form of an animal feed, a health supplement or a nutrient supplement.

In an embodiment of the present disclosure, there is provided a composition comprising an organic selenium enriched edible marine microalga Nannochloropsis oceanica as described herein, wherein the composition is used in a form of an animal feed, a health supplement or a nutrient supplement; and wherein organic selenium enriched edible marine microalga Nannochloropsis oceanica is present in the range 10-30% (w/w).

In an embodiment of the present disclosure, there is provided a process for obtaining an organic selenium enrichment of edible marine microalga Nannochloropsis oceanica CASA CC201 as described herein, wherein the organic selenium enriched edible marine microalga Nannochloropsis oceanica CASA CC201 is treated with inorganic selenium, wherein the process enhances biomass productivity.

In an embodiment of the present disclosure, there is provided an organic selenium enriched edible marine microalga Nannochloropsis oceanica CASA CC201 as disclosed herein, wherein the organic selenium enriched edible marine microalga Nannochloropsis oceanica CASA CC201 is treated with inorganic selenium, and wherein the process enhanced bioactivity.

In an embodiment of the present disclosure, there is provided an organic selenium enriched edible marine microalga Nannochloropsis oceanica CASA CC201 as described herein, wherein the edible marine microalga is an indigenous producer of omega 3 fatty acid.

In an embodiment of the present disclosure, there is provided a micronutrient enriched edible microalgal biomass, comprising at least 99% of an organic selenium, along with omega 3 fatty acid in the range of 9-12% and MUFA in the range of 33-35%, wherein the micronutrient enriched edible microalgal biomass is capable of withstanding up to 1000 μM sodium selenite without any lag in its growth.

In an embodiment of the present disclosure, there is provided an organic selenium enriched edible marine microalga Nannochloropsis oceanica CASA CC201 for use in treating dietary selenium deficiency, wherein the organic selenium enriched edible marine microalga is used as food or feed additive.

In an embodiment of the present disclosure, there is provided a composition comprising an organic selenium enriched edible marine microalga Nannochloropsis oceanica in the range 10-30% (w/w), wherein the composition is in form of a food additive. In another embodiment of the present disclosure, the composition is in form of a feed additive. In yet another embodiment of the present disclosure, the composition is in form of a health supplement. In one another embodiment of the present disclosure, the composition is in form of a nutrient supplement.

In another embodiment of the present disclosure, there is provided a composition comprising an organic selenium enriched edible marine microalga Nannochloropsis oceanica as described herein, for use in treating dietary selenium deficiency.

In an embodiment of the present disclosure, there is provided a method of treating dietary selenium deficiency, said method comprising: administering the organic selenium enriched edible marine microalga Nannochloropsis oceanica as described herein to a subject in need thereof.

In an embodiment of the present disclosure, there is provided a method of treating dietary selenium deficiency, said method comprising: administering composition comprising an organic selenium enriched edible marine microalga Nannochloropsis oceanica as described herein, to a subject in need thereof.

### Acronyms Used to Describe the Invention:

GC MS: Gas Chromatography Mass Spectrometry

SEM: Scanning Electron Microscopy

ICP MS: Inductively coupled plasma mass spectrometry

Se: Selenium

Nanno Se⁺: 500 μM Na₂SeO₃ acclimatized N. oceanica CASA CC201; organic selenium enriched

Nanno Se⁺⁺: Nanno Se⁺ when transferred to fresh medium containing 500 μM Na₂SeO₃; organic selenium enriched

Nanno Se*: High selenium tolerant N. oceanica CASA CC201

Nanno Se⁻: Nanno Se⁺ when transferred to fresh medium without selenium-organic selenium enriched

EPA: Eicosapentaenoic acid

SFA: Saturated fatty acid

PUFA: Poly Unsaturated Fatty Acid

MUFA: Mono Unsaturated Fatty Acid

Na₂SeO₃: Sodium selenite

N. oceanica: Nannochloropsis oceanica CASA CC201

μM: Micromolar

DW: Dry weight

### Material and Method Used in Experiments:

Materials Required:

For the purpose of the present disclosure, marine microalga Nannochloropsis oceanica CASA CC201 was obtained from CSIR-CSMCRI Marine Algal Research Statio Mandapam Camp, Ramanathapura District, Tamil Nadu—PIN 623519.

Methods:

(1) Maintenance of Microalga N. oceanica CASA CC201:

Marine microalga Nannochloropsis oceanica CASA 201 were cultured in standard D Walne's medium. For the purpose of the present invention, Marine microalga Nannochloropsis oceanica CASA 201 were cultured in 1 L conical flask with 400 ml D Walne's medium and were maintained at 25±2° C. and the culturing was done during 14:10 hours light-dark period. The cultures were shaken intermittently in order to keep away the cells from attaching to the walls of the flask and settling down of cells.

(2) Inorganic Selenium Treatment

For the purpose of developing the organic selenium enriched N. oceanica CASA 201, the strain N. oceanica CASA 201 was inoculated in to a fresh D Walne's medium (10% v/v of log phase culture)), and the culture was subsequently subjected to different concentrations of inorganic selenium, i.e., sodium selenite. Sodium selenite (Na₂SeO₃) was added at the time of inoculation.

Different concentrations of the inorganic selenium were prepared by following process: 1 M sodium selenite (HiMedia, India) stock was prepared by dissolving 17.29 g sodium selenite in 100 ml autoclaved distilled water to obtain the solution and the solution was sterilized by filter sterilization using 0.22 μM nylon membrane filter (HiMedia, India). From 1M stock solution, different volumes of sodium selenite were taken and added to each flask so as to get the corresponding concentrations using the formula;

N₁V₁=N₂V₂

N₁—1M sodium selenite stock
V₁—Volume of stock to be added to each flask to attain the desired sodium selenite concentrations.
N₂—Desired concentration of sodium selenite
V₂—volume in each flask (400 ml)

For the experimentation purpose, the culture without any sodium selenite served as the control.

(3) Growth Analysis

In order to monitor the growth, cell count at different time intervals was monitored by taking 1 ml sample from each flask at 3 days interval by a Neubauer counting chamber under microscope (LEICA DM-2000 MODEL). Growth in terms of biomass productivity was done by measuring the dry weight at 3 days intervals by lyophilizing the harvested culture.

(4) Selenium Quantification by ICP MS Analysis

For the ICP MS analysis, the cultures were harvested by centrifugation at 8000 rpm for 15 minutes. The cultures were then washed 3 times with sterile distilled water and lyophilized subsequently.

Total selenium was analysed by the process as follows:
(a) 100 mg of lyophilized sample was taken in digestion vessel and added with 5 ml of 65% HNO₃ (Merck, supra pure) and 2 ml of H₂O₂. Microwave-assisted digestion was implemented using:
(i) 15 min ramp till 130° C. from ambient and held for 2 minutes at 800 W applied power.
(ii) Further the temperature was increased to 185° C. in a 10 min ramp time and kept the samples at 185° C. at 30 min at 800 W applied power.
(b) After digestion the samples were diluted to 50 ml and ICP MS analysis was done.

Further. The inorganic selenium was analysed by the process as follows: 100 mg sample was extracted with 15% HCl (Merck, supra pure) and the extract was analysed directly.

Organic selenium was calculated by subtracting inorganic selenium content from total selenium content.

(5) Scanning Electron Microscopy

SEM analysis was carried out for the morphological analysis of inorganic selenium treated and untreated N. oceanica CASA CC201. The treated and untreated microalgal cells were fixed with 2% glutaraldehyde in 1M phosphate buffer for overnight. The cells were washed thoroughly with phosphate buffer (pH 7) to remove the excess glutaraldehyde attached to it. The samples were then added on clean cover slip and dehydrated with varying concentrations of alcohol ranging from 50 to 100%. The cover slip with sample placed on a metal stub and then sputter coated with gold. The SEM images were taken at 15 kV voltage and 1500KX magnification.

(6) Lipid Extraction and Fatty Acid Analysis by GCMS

Sodium selenite treated and untreated N. oceanica CASA CC201 biomass were harvested by centrifugation at 8000 rpm for 15 min and lyophilized. In order to disrupt the cell and to release the lipids, 2 ml of chloroform:methanol (2:1) and 2 ml of sodium chloride were added to the lyophilized microalgal biomass and mixed thoroughly by vortexing. The lipid containing phase was collected by centrifuging the sample at 10000 rpm for 15 min. After centrifugation, the lipids were present in the lower phase and it was transferred to pre weighed 50 ml round bottom (RB) flask. Evaporation of the solvents in the lower phase was done by using a rotavapour (Buchi). In order to make the extracted lipids more volatile and also to protect the column from acid attack, the total lipids were transesterified with methanol prior to gas chromatography. The transesterification was done by adding 2% H₂SO₄ in dried methanol to the total lipid and heated for 1 hr in a water bath at 100° C. After 1 hr, it was cooled, and 3 ml hexane was added to it. The mixture was then vortexed thoroughly for 1 min and centrifuged at 10000 rpm for 5 min. The upper layer was taken, diluted with hexane and filtered. The filtrate was then subjected to gas chromatography.

### EXAMPLES

Following are the examples given to further illustrate the invention and should not be construed to limit the scope of the present disclosure. Unless defined otherwise, all technical and scientific terms used herein have the same meaning as commonly understood to one ordinary person skilled in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

In the present disclosure, a bioprocess was developed for production of organic selenium enriched edible marine microalga Nannochloropsis oceanica CASA CC201.

Following Scheme-1 provides representation of organic selenium enrichment in edible microalga N. oceanica by using inorganic selenium.

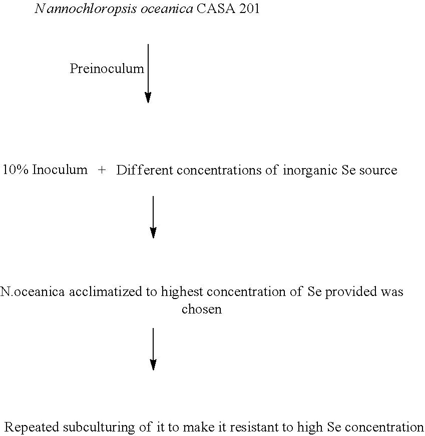

The forthcoming examples explain the process of treating N. oceanica with different concentrations of inorganic selenium was performed in order to make the organism acclimatized to its growing environment. The culture medium used was D Walne's. Filter sterilized (0.2 μm) 1 Molar sodium selenite as the stock. Different concentrations of sodium selenite were added to each experimental flask containing sterilized medium prior to inoculation. 10% mid log phase inoculum was used.

The fatty acid content in the selenium acclimatized N. oceanica was analyzed by Gas Chromatography-Mass spectrometry (GC-MS) to analyze the Poly Unsaturated Fatty Acid (PUFA) like Eicosapentanoic acid (EPA) and other PUFA as well, namely C18:2 (Linoleic acid; PUFA); C20:4 (Arachidonic acid, PUFA) and two other MUFA content. All the experiments as described below were conducted in triplicate.

Since the organic selenium enriched edible marine microalga comprises more than 99% of an organic selenium, along with omega 3 fatty acid in the range of 9-12% and MUFA in the range of 33-35%, therefore, it is used as a food or feed additive to ensure adequate dietary selenium requirement.

### Example-1: Treatment of Edible Marine Microalga Nannochloropsis oceanica CASA 201 with Different Concentrations of Inorganic Sodium Selenite (Sodium Selenite, Na₂SeO₃)

In order to enrich edible marine microalga Nannochloropsis oceanica CASA 201 with organic selenium, initially the organism was made tolerable to inorganic Se. For that, the microalga was exposed to different concentration (100-500 μM) of inorganic selenium in the form of Sodium selenite, Na₂SeO₃.

### Example-2: Effect of Externally Added Sodium Selenite on the Growth of Nannochloropsis Oceanica CASA 201

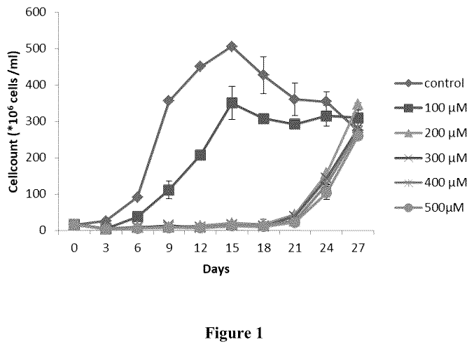

The growth of the cells was monitored by taking cell count at 3 days interval. The culture exposed to high Se concentration stopped their growth after 3 days. The growth of the culture which exposed to lowest Se concentration was diminished as compared to control even though they can grow. After 21^{st} day, the cells which are acclimatized to higher concentrations of sodium selenite started their growth. FIG. 1 illustrates growth of N. oceanica CASA CC201 exposed to different concentrations of sodium selenite (Na₂SeO₃) ranging from 0 (control)-500 μM. Upon external exposure to inorganic Se, growth was observed only in 100 μM sodium selenite concentration and it was slow when compared to control. But from 21^{st} day onwards, 200-500 μM sodium selenite exposed cultures started their growth.

### Example-3: Quantification of Intracellular Selenium Content in Nannochloropsis oceanica CASA 201 by ICP MS

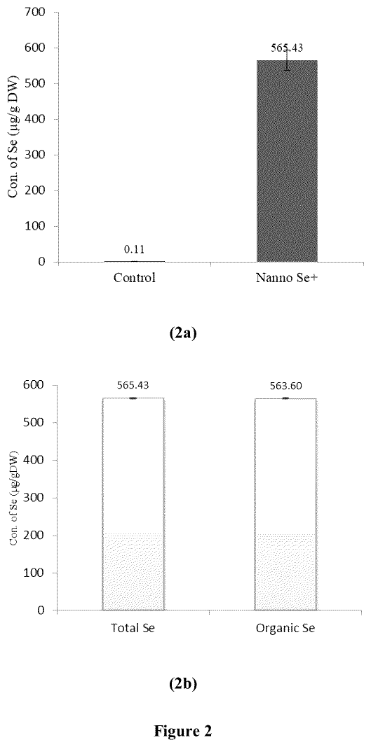

On 31^{st} day, the total selenium and organic selenium in control as well as 500 μM Na₂SeO₃ acclimatized N. oceanica CASA CC201 (Nanno Se⁺) were analysed by ICP MS. The total selenium content in Nanno se was 565.43 μg/g DW, whereas in control it was 0.11 μg/g DW (FIG. 2a). The total selenium content which includes both inorganic and organic forms were very high in Se treated Nannochloropsis oceanica CASA 201 as compared to control where no external selenium was added. Since the organic form of selenium was more absorbable and digestible and also less toxic than inorganic form. The organic selenium content in Nanno Se⁺ was found to be 563.60 μg/g DW in the total selenium content of 565.43 μg/g DW (FIG. 2b). Around 99% of total intracellular selenium was found to be organic selenium.

FIG. 2a &b is an illustration of selenium quantification by ICP MS. (a) Total intracellular Se content in untreated N. oceanica CASA CC201 (control) and 500 μM Na₂SeO₃ acclimatized N. oceanica CASA CC201 (Nanno Se⁺). Intracellular Se content was found to 565.43 μg/g biomass. (b) Total Se and Organic Se content in 500 μM Na₂SeO₃ acclimatized N. oceanica CASA CC201 (Nanno Se⁺). Organic Se enrichment was observed in Nanno Se⁺.

### Example-4: Morphology Analysis of Nannochloropsis oceanica CASA 201 by Scanning Electron Microscopy (SEM)

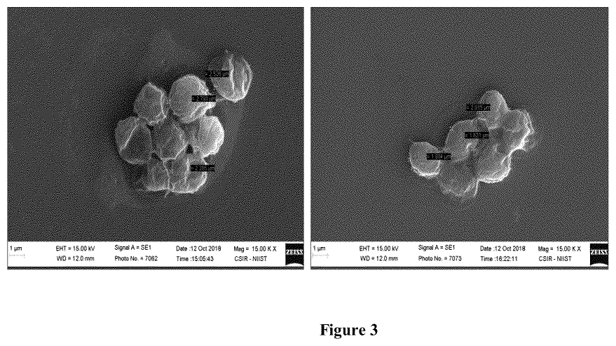

Organic selenium enriched Nannochloropsis oceanica CASA 201 (Nanno Se⁺) was observed under Scanning Electron Microscope to check whether any morphological changes occurred when they were exposed to stress like external inorganic selenium at high concentration as compared to normal cells. SEM images show that even though there was a size reduction in Nanno Se⁺, the difference was negligible. FIG. 3 illustrates morphology analysis of untreated (a) and Nanno se (b) by SEM. No significant changes observed.

### Example-5: Analysing the Tolerance of Selenium Acclimatized Nannochloropsis oceanica CASA 201 by Exposing them Again to Inorganic Selenium

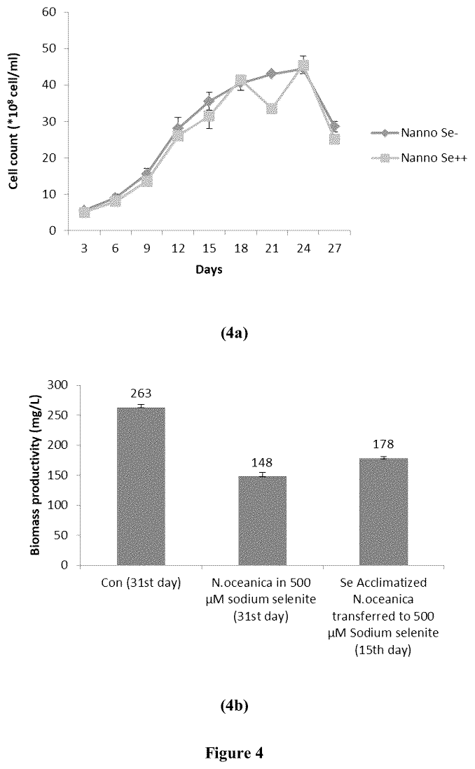

In order to increase the tolerance, to reduce the lag phase and to increase the biomass concentration, Nanno Se⁺ was harvested by centrifugation at 8000 rpm for 15 minutes and transferred to fresh medium with sodium selenite (Nanno Se⁺⁺) and without sodium selenite (Nanno Se⁻). The growth was monitored by taking the cell count at 3 days interval. The selenium acclimatized Nannochloropsis oceanica CASA 201 can grow without any lag in fresh medium in presence and absence of selenium (FIG. 4a). The biomass productivity was also analysed. When the highest Se acclimatized Nannochloropsis oceanica CASA 201 was exposed to the same Se concentration again, the biomass productivity was increased. On 31^{st} day only 148±7 mg/L biomass productivity was observed. But when the acclimatized cultures were transferred to fresh medium containing same Se concentration, the biomass productivity increased to 178±3 mg/L within 15 days (FIG. 4b). The subsequent exposure of Se acclimatized N. oceanica again to inorganic selenium made them tolerant to the high concentration and were able to reduce the lag phase in their growth.

FIG. 4a illustrates growth of Se acclimatized N. oceanica CASA CC201 when transferred to fresh medium with 500 μM Na₂SeO₃ (Nanno Se⁺⁺) and without Na₂SeO₃ (Nanno Se⁻). Both of them started their growth without any lag. FIG. 4b illustrates biomass productivity of N. oceanica CASA CC201 under Se treatment on 31^{st} day and when the Se acclimatized culture transferred to fresh medium with 500 μM Na₂SeO₃ (15^{th} day). Biomass productivity was found to be low in 500 μM Na₂SeO₃ exposed N. oceanica CASA CC201 (Nanno Se⁺) on 31^{st} day when compared to control. But when transferred to fresh medium containing 500 μM Na₂SeO₃, biomass productivity has increased within 15^{th} day.

### Example-6: Quantification of Intracellular Selenium Content in Se Acclimatized Nannochloropsis oceanica CASA 201 Growing in Sodium Selenite with and without Medium by ICP MS

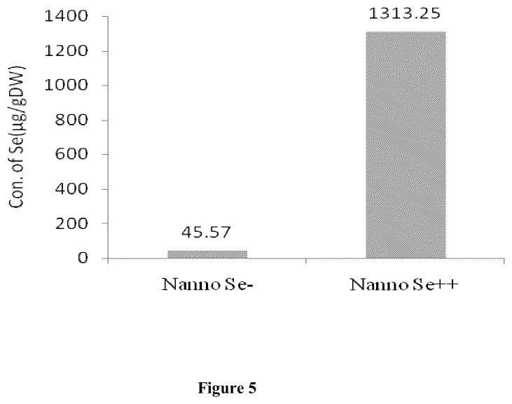

On 15^{th} day, Nanno Se⁺⁺ and Nanno Se⁻ (Nanno Se⁺) when transferred to fresh medium with and without selenium) were harvested for ICP MS analysis. The total selenium content was quantified (FIG. 5). In Nanno Se⁻, the total intracellular Se content was found to be 45.57 μg/g where in, Nanno Se⁺⁺, the total intracellular Se content was found to be 1313.25 μg/g. FIG. 5 illustrates Selenium quantification in Se acclimatized N. oceanica CASA CC201 (Nanno Se⁺) when transferred to fresh medium containing 500 μM Na₂SeO₃ (Nanno Se⁺⁺) and without Na₂SeO₃ (Nanno Se⁻) by ICP MS. A significant enrichment in selenium content was observed in Nanno Se⁺⁺.

### Example-7: Analysis of Fatty Acid Content in Organic Selenium Enriched Nannochloropsis Oceanica CASA 201

Since Nannochloropsis oceanica CASA 201 is known for its oleaginous nature with notable amount of Eicosapentanoic acid (EPA) content, the fatty acid profile of Nannochloropsis oceanica CASA 20/(control), Nanno Se⁺, Nanno Se⁻ and Nanno Se⁺⁺ was checked by GC MS to know whether there any significant change in their fatty acid profile (Table: 1). In control, among the total fatty acids saturated fatty acids (SFA) constituted around 50% and unsaturated fatty acids found to be present around 49.17%. In Nanno Se⁺, SFA content was around 51.37% and unsaturated fatty acids were 48.63%. In control, the unsaturated fatty acids constituted of 31.97% and 17.20% of monounsaturated (MUFA) and polyunsaturated (PUFA) fatty acids respectively. The EPA content was around 11.17% of PUFA. In Nanno Se⁺, the unsaturated acid constituted of 33.60% and 15.03% of MUFA and PUFA respectively. EPA content was 9.54%. There was a slight variation in the ratio of MUFA and PUFA in Nanno Se⁺ as compared to control. MUFA content was found to be slightly increased whereas PUFA content slightly decreased as a result of Se treatment.

When Nanno Se⁺ were transferred to fresh medium with and without Se, SFA content showed a slight reduction in their proportion as compared to unsaturated fatty acid. SFA content was found to be 49.55% in Nanno Se⁻ and 48.77% in Nanno Se⁺⁺. Unsaturated fatty acid content in Nanno Se⁻ was 50% (MUFA; 30% and PUFA; 20%) and in Nanno Se⁺⁺, unsaturated fatty acid was 51.23% (MUFA; 35.94% and PUFA; 15.29%). EPA content was 9.065%.

TABLE 1
Analysis of fatty acid content in selenium enriched N. oceanica

| | Control | Nanno Se⁺ | Nanno Se⁻ | Nanno Se⁺⁺ |
|---|---|---|---|---|
| Fatty acid (%) | (31st day) | (31st day) | (15th day) | (15th day) |
| C14:0 | 6.76 ± 0.37 | 5.62 ± 0.252 | 7.07 ± 0.27 | 6.36 ± 0.145 |
| (Myristic acid; SFA) | | | | |
| C16:0 | 38.76 ± 2.63 | 27.3 ±1.236 | 37.87 ± 0.65 | 38.71 ± 0.075 |
| (Palmitic acid; SFA) | | | | |
| C16:1 | 21.94 ± 0.704 | 25.19 ± 1.584 | 21.55 ± 0.13 | 25.4 ± 0 |
| (Palmitoleic acid; MUFA) | | | | |
| C18:0 | 2.55 ± 0.343 | 2.67 ± 0.708 | 3.145 ± 0.025 | 2.645 ± 0.157 |
| (Stearic acid; SFA) | | | | |
| C18:1 | 10.03 ± 0.582 | 8.23 ± 0.075 | 8.485 ±0.355 | 10.035 ± 0.465 |
| (Elaidic acid; MUFA) | | | | |
| C18:2 | 2.2 ± 0.347 | 2.03 ± 0.145 | 3.535 ± 0.105 | 2.52 ± 0.15 |
| (Linoleic acid; PUFA) | | | | |
| C20:4 | 3.83 ± 0.627 | 3.45 ± 0.209 | 3.75 ± 0.16 | 3.545 ± 0.075 |
| (Arachidonic acid, PUFA) | | | | |
| C20:5 | 11.17 ± 3.25 | 9.54 ± 1.22 | 13.155 ± 0.405 | 9.065 ± 0.145 |
| (Eicosapentaenoic acid, PUFA) | | | | |
| SFA | 50.8 ± 3.744 | 51.37 ± 1.321 | 49.55 ± 0.165 | 48.77 ± 0.165 |
| MUFA | 31.97 ± 1.045 | 33.60 ± 1.475 | 30.0 ± 0.44 | 35.94 ± 0.44 |
| PUFA | 17.20 ± 4.058 | 15.03 ± 1.225 | 20.1 ± 0.405 | 15.29 ± 0.53 |

### Example-8: Se Acclimatized N. oceanica in Higher Concentrations of Sodium Selenite Exposed to More Se to Make them Tolerable to High Inorganic Selenium and to Enrich with High Intracellular Organic Selenium Content

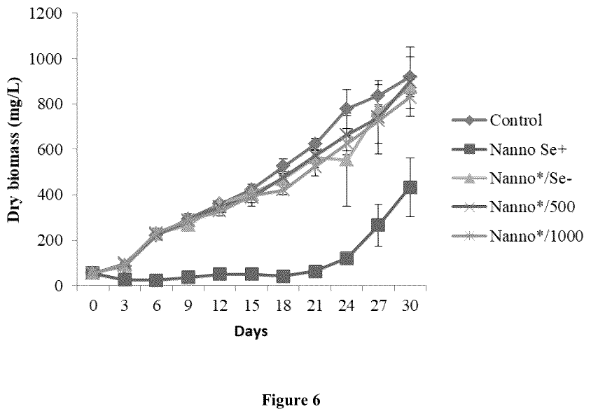

Nanno Se⁺⁺ was again subcultured in much more Se concentrations of inorganic Se containing medium and in between Se free medium to make them tolerable to high inorganic selenium and to enrich with high intracellular organic selenium content. Nanno Se⁺⁺ is now termed as Nanno Se*. Nanno Se* was now able to grow in medium containing high Se concentrations (1000 μM) which are comparable to control. FIG. 6 illustrates growth of high selenium tolerant N. oceanica CASA CC201 (Nanno*) with respect to control in terms of biomass productivity. No reduction in biomass productivity observed in Nanno* under exposure to different concentrations of sodium selenite.

### ADVANTAGES OF THE PRESENT DISCLOSURE

The present disclosure discloses an organic selenium enriched edible marine microalga Nannochloropsis oceanica CASA CC201, which is edible, and enriched with organic selenium.

Organic selenium is more absorbable and digestible than inorganic selenium and less toxic also.

Since the organic selenium enriched edible marine microalga Nannochloropsis oceanica CASA CC201 which is rich with EPA increases its product value, and therefore, it is used as food or feed additive.

The organic selenium enriched edible marine microalga is cultured in natural sea water. Thus, helps to reduce its production cost as sea water is abundantly available and it contains required nutrients.

Further, less dependence of precious/limiting agriculture input fresh water required for large scale commercial cultivation.

1. An organic selenium enriched edible marine microalga Nannochloropsis oceanica CASA CC201.

2. The organic selenium enriched edible marine microalga Nannochloropsis oceanica CASA as claimed in claim 1, wherein the organic selenium enriched edible marine microalga Nannochloropsis oceanica CASA comprises at least 99% of organic selenium, along with omega 3 fatty acid in the range of 9-12%, and monounsaturated fatty acid (MUFA) in the range of 33-35%.

3. The organic selenium enriched edible marine microalga Nannochloropsis oceanica CASA as claimed in claim 1, wherein the organic selenium enriched edible marine microalga Nannochloropsis oceanica CASA further comprises EPA content in the range of 9-12%.

4. A process for obtaining an organic selenium enriched edible marine microalga Nannochloropsis oceanica CASA CC201 as claimed in claim 1, said process comprises treating N. oceanica CASA CC201 with an inorganic selenium having a concentration in the range of 100-500 μM over several generations for obtaining an organic selenium enriched edible marine microalga Nannochloropsis oceanica CASA CC201.

5. The process as claimed in claim 4, said process further comprising:
i. treating N. oceanica CASA CC201 with an inorganic selenium having a concentration in the range of 100-500 μM over several generations for enrichment of organic selenium;
ii. transferring N. oceanica CASA CC201 of step (i) to a fresh medium with inorganic selenium and without inorganic selenium to know whether it can really tolerate the high inorganic selenium; and
iii. enriching N. oceanica CASA CC201 which can withstand the highest concentration of inorganic selenium in the same manner as enriched in step (ii) for obtaining an organic selenium enriched edible marine microalga Nannochloropsis oceanica CASA CC201.

6. The process as claimed in claim 5, wherein said process optionally comprises transferring N. oceanica CASA CC201, which can tolerate maximum inorganic selenium, to more high inorganic selenium concentration in the range of 700-1000 μM to enrich the intracellular organic selenium content and to enhance biomass productivity.

7. The process as claimed in claim 4, wherein the inorganic selenium is sodium selenite.

8. The process as claimed in claim 4, wherein the organic selenium enriched edible marine microalga Nannochloropsis oceanica CASA is cultured in natural sea water.

9. A composition comprising the organic selenium enriched edible marine microalga Nannochloropsis oceanica as claimed in claim 1.

10. The composition as claimed in claim 9, wherein the organic selenium enriched edible marine microalga Nannochloropsis oceanica is present in the range 10-30% (w/w).

11. The composition as claimed in claim 9, said composition is used in a form of an animal feed, a health supplement, or a nutrient supplement.
